(19) United States
(12) Patent Application Publication
(10) Pub. No.: US 2025/0143986 A1
(43) Pub. Date: May 8, 2025

(54) COMPOSITION FOR MICRONEEDLE PREPARATION, COMPRISING CYCLODEXTRIN FOR RETINOL STABILIZATION, AND MICRONEEDLE PREPARED WITH SAME COMPOSITION

(71) Applicant: RAPHAS CO., LTD., Seoul (KR)

(72) Inventors: Yee-Ram CHOI, Seoul (KR); Joo-Hyeon JANG, Seoul (KR); Sung-Su KIM, Anyang-si, Gyeonggi-do (KR); Gwon-Woo KIM, Seoul (KR); Boo-Yong LEE, Gimpo-si, Gyeonggi-do (KR); Keun-Ho LEE, Goyang-si, Gyeonggi-do (KR); Jung-Hyun BAE, Seoul (KR); Chang-Yub SUNG, Bucheon-si, Gyeonggi-do (KR); Soo-Kie LA, Seoul (KR); Hyun-Woo PARK, Seoul (KR); Do-Hyeon JEONG, Seoul (KR)

(73) Assignee: RAPHAS CO., LTD., Seoul (KR)

(21) Appl. No.: 18/287,455

(22) PCT Filed: Apr. 11, 2022
(86) PCT No.: PCT/KR2022/005179

§ 371 Date: Aug. 12, 2024

(30) Foreign Application Priority Data
Apr. 19, 2021 (KR) 10-2021-0050307

(51) Int. Cl. A61K 8/34; A61K 8/02; A61K 8/73; A61Q 19/08

(52) U.S. Cl. CPC A61K 8/342; A61K 8/0204; A61K 8/735; A61K 8/738; A61Q 19/08; A61K 2800/91

(57) ABSTRACT

The present invention relates to a retinol-loaded microneedle. The retinol-loaded microneedle according to the present invention contains retinol and a stabilizer therefor, which is an optimal combination for use in microneedles, in order to solve the instability of retinol. Accordingly, the retinol-loaded microneedle of the present invention achieves high transdermal delivery efficiency intrinsic for microneedle formulations and thus can maximize the effect of retinol on wrinkle reduction, etc.

### TECHNICAL FIELD

The present disclosure relates to a stabilization method of retinol for application to a microneedle formulation, and more particularly, to a composition for manufacturing a microneedle including cyclodextrin for stabilization of retinol, and a microneedle manufactured using the composition.

### BACKGROUND ART

Retinol is known to have an effect of reducing wrinkles and increasing skin elasticity by promoting differentiation of skin cells and promoting biosynthesis of collagen, which affects wrinkles, and elastin, which affects elasticity (refer to the document [Reza Kafi, MD; Heh Shin R. Kwak, MD; Wendy E. Schumacher, B S; et al. Arch Dermatol. 2007; 143(5):606-612]). However, retinol has a double bond structure, and thus is easily oxidized to form free radicals, or becomes unstable when exposed to ultraviolet rays, changing from a trans form to a cis form (refer to the document [Int. J. Environ. Res. Public Health 2005, 2(1), 147-155]]. When retinol is oxidized due to an influence of an external environment to form free radicals or change to an unstable cis form, there is a problem in that retinol does not have inherent effects thereof, such as wrinkle reduction, and may actually have a negative effect on the skin.

Despite definite effects such as wrinkle reduction, retinol is very unstable and it is difficult to develop technologies to stabilize retinol, and thus a few cases have succeeded in commercializing retinol.

Microneedles are a delivery system for active ingredients that combines the efficacy of existing syringes and the convenience of patches and are attracting attention because microneedles are capable of delivering active ingredients directly into the skin through the stratum corneum, the skin barrier layer. When retinol is loaded in microneedles, delivery efficiency may be greatly improved, and thus when the stability of the loaded retinol is ensured, it is expected to exhibit much better efficacy than retinol products in the form of emulsions such as oil-in-water and water-in-oil that are currently on the market.

Therefore, there is a need for a formulation for improving the stability of retinol when retinol is manufactured in a microneedle form.

### DISCLOSURE

### Technical Problem

Accordingly, the present inventors make diligent efforts to develop a microneedle and stabilization formulation for improving the stability of retinol, and as a result, it is seen that a microneedle with greatly improved stability of retinol is obtained when cyclodextrin is used as a stabilizer, completing the present disclosure.

### Technical Solution

To achieve the aforementioned objectives, an aspect of the present disclosure provides a composition for manufacturing a microneedle, including retinol or a derivative thereof, and a biodegradable solidified material.

Another aspect of the present disclosure provides a dissolvable microneedle manufactured using the composition.

Another aspect of the present disclosure provides a patch including the microneedle.

### Advantageous Effects

According to the present disclosure, to overcome the instability of retinol, a retinol-loaded microneedle includes retinol and its stabilizer, which is an optimal combination for use in a microneedle. Accordingly, the retinol-loaded microneedle according to the present disclosure may achieve high skin delivery efficiency dedicated to microneedle formulation, thereby maximizing an effect of retinol, such as wrinkle reproduction.

### DESCRIPTION OF DRAWINGS

FIG. 1 is a graph showing the results of a 4-week accelerated test on a retinol raw material candidate to be loaded in a microneedle.

FIG. 2 is a graph showing the results of a stability test (accelerated+long term) for 4 weeks on microneedle samples manufactured using tocopherol as a stabilizer for retinol.

FIG. 3 is a graph showing the results of a stability test (accelerated+long term) for 4 weeks on microneedle samples manufactured using sodium ascorbate as a stabilizer for retinol.

FIG. 4 is a graph showing the results of a stability test (accelerated) for 8 weeks on microneedle samples manufactured using cyclodextrin as a stabilizer for retinol.

FIG. 5 is the result of an experiment evaluating the stability of the content of cyclodextrin contained in a microneedle.

### BEST MODE

Hereinafter, the present disclosure will be described in more detail.

One aspect of the present disclosure provides a composition for manufacturing microneedles, including retinol or a derivative thereof, cyclodextrin, and a biodegradable solid material.

The term “retinol” used in the specification refers to vitamin A, one of the fat-soluble vitamins, and a necessary ingredient for normal differentiation of cells and growth of bones, teeth, hair, and nails. In particular, retinol is known to have excellent antioxidant activity and have an effect of reducing wrinkles and increasing skin elasticity by promoting differentiation of skin cells and promoting the biosynthesis of collagen, which affects wrinkles, and elastin, which affects elasticity.

The term “retinol derivative” used in the specification refers to a compound obtained by modifying retinol to improve the functionality and stability of retinol. Examples of retinol derivatives may include, but are not limited to, retinol esters (e.g., retinyl palmitate, retinyl acetate, and retinyl propionate), retinoic acid, retinol aldehyde, and retinal.

In one embodiment of the present disclosure, commercially available retinol raw materials are used. As described later, a total of four commercially available retinol raw materials (hereinafter referred to as A to D) are used as retinol raw material candidates. Among these, a retinol raw material with relatively high stability, such as A, is selected and used in a retinol stabilization experiment. However, a retinol stabilization method or retinol stabilization agent dedicated to the present disclosure does not need to be accepted as being effective only for specific retinol raw materials. A person skill in the art may easily attempt retinol stabilization using a plurality of commercially available retinol raw materials in the same manner as the teachings of the present disclosure and may understand the same effect. Therefore, any retinol raw material used is included in the scope of the retinol stabilization method or agent of the present disclosure.

The biodegradable solidified material may be any material without limitation as long as the material forms a microneedle, maintains a shape thereof, and dissolves in the body after penetrating the skin. In detail, the biodegradable solidified material may be a hydrophilic material. The biodegradable solidified material includes, for example, hyaluronic acid (HA) or a pharmaceutically acceptable salt thereof, carboxymethyl cellulose, polyvinylpyrrolidone, polyvinyl alcohol, polylactic glycolic acid, gelatin, collagen, chitosan, or a mixture thereof, but is not limited thereto.

In one embodiment of the present disclosure, the biodegradable solidified material may be hyaluronic acid (HA) or a pharmaceutically acceptable salt thereof. An average molecular weight of hyaluronic acid (HA) may be 10 to 5000 kDa, in detail, 40 kDa to 150 kDa.

Hyaluronic acid (HA) is a bio-derived polymer including N-acetyl-D-glucosamine and D-glucuronic acid that are combined in alternating chains, is abundant in animal tissues such as the skin and the umbilical cord, is used as a biomaterial, and has physical properties that are easily controlled.

Another aspect of the present disclosure provides a dissolvable microneedle manufactured using the composition.

The term “microneedle” used in this specification refers to a needle-shaped structure with a length in micrometers (μm), and a tip thereof has a pointed shape like a needle, allowing the microneedle penetrate the skin. The microneedle forms a hole in the stratum corneum, the outermost layer of the skin, and delivers drugs through the hole formed as such. In addition, the microneedle is very short and does not affect nerve cells, and thus rarely causes pain.

As used in the specification, the term “dissolvable microneedle” refers to a microneedle that releases loaded drugs by dissolving in the body when applied to the skin. In the case of a non-soluble hydrogel type microneedle, the skin swells when the microneedle is applied to the skin, which may cause irritation by putting pressure on the skin and may cause pain during a removal process. However, in the case of the dissolvable microneedle, the dissolvable microneedle disappears after being applied to the skin, and thus does not need to be removed.

In one embodiment of the present disclosure, the microneedle may be manufactured using a droplet extension (DEN) method.

The DEN method refers to a scheme of simultaneously manufacturing two microneedle patches by dropping droplets including an active ingredient on one patch, allowing the patch to be in contact with an opposite patch and extending the patches to form a microneedle, solidifying the resulting material through blowing, and then separating the resulting material. This is a unique scheme suitable for loading biopharmaceuticals due to a simple manufacturing process and excellent mass production compared with a molding method as the existing method of manufacturing a microneedle.

For the microneedle manufactured using the DEN method, reference may be made to Korean patent Nos. 1254240, 1285085, 1636069, 1816922, 2103194, and 2127123, and the full contents of these patents are incorporated herein by reference.

According to one embodiment of the present disclosure, the microneedle may be used to reduce wrinkles or increase skin elasticity.

Another aspect of the present disclosure provides a patch including the microneedle.

The term “patch” used in the specification refers to a formulation that is attached to the skin and delivers drugs into the body.

Hereinafter, the present disclosure will be described in more detail through the following examples. However, the following examples are only for illustrating the present disclosure, and the scope of the present disclosure is not limited to these only.

### Example 1. Select Retinol Raw Material for Microneedle

To select a retinol raw material with excellent stability when formulated into a microneedle, a stability test was performed using four commercially available retinol raw materials A, B, C, and D as candidates.

For ingredients of the four retinol raw material candidates, reference may be made to Table 1 below.

TABLE 1

| Retinol raw | |
|---|---|
| material | Ingredients* |
| A | Glycine soja (soy bean) oil, retinol |
| B | Caprylic/capric triglyceride, sodium ascorbate, tocopherol, |
| | retinol |
| C | Water, Glycine Soja (soy bean) oil, PEG-4, retinol, |
| | behenamidopropyl dimethylamine behenate, behenic acid |
| D | water, butylene glycol, glycine soja (soy bean) oil, retinol, |
| | pentylene glycol, carbomer, tocopherol, acacia senegal |
| | gum, propylene glycol alginate |

*Ingredients are based on CTFA On-Line database.

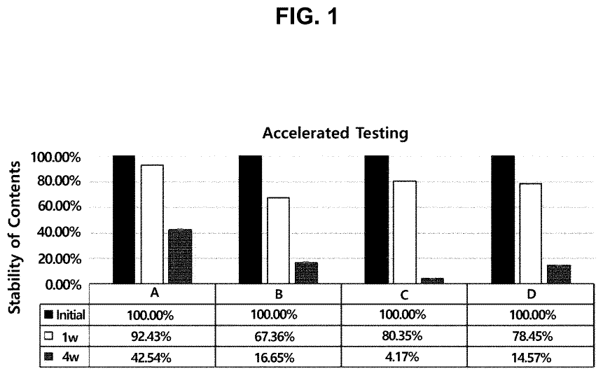

The four retinol raw materials were each put and sealed in an aluminum pouch used to pack microneedle formulation and then exposed to an accelerated condition (40±2° C., relative humidity 75±5%) for 4 weeks to perform an accelerated test. The content stability of retinol was evaluated at first, second, and fourth weeks. As a result, it was seen that retinol raw material candidate A had relatively high stability (FIG. 1). Therefore, retinol raw material A was selected as a retinol raw material for loading a microneedle.

### Example 2. Evaluate Retinol Stabilizer with Microneedle Formulation

Four stabilizers shown in Table 2 below were selected as stabilizer candidate materials. The main physicochemical functions of the stabilizer candidate materials are listed in Table 2.

TABLE 2

| Function | Name | Main physicochemical function |
|---|---|---|
| Stabilizer | Butylated | Ingredient that acts as oxygen scavenger |
| | Hydroxytoluene | and prevents cosmetics from deteriorating. |
| | (BHT)) | fat-soluble antioxidant |
| | | Ingredient that may cause hives on |
| | | sensitive skin |
| | Tocopherol | Vitamin E, fat-soluble vitamin, and natural |
| | | antioxidant isolated from vegetable oil. |
| | | All substances containing unsaturated |
| | | groups protect other materials from |
| | | oxidation while being oxidized. |
| | Sodium ascorbate | Sodium salt of ascorbic acid |
| | | Ingredient that protects double bonds and |
| | | acts as an oxygen scavenger to prevent |
| | | oxidation. |
| | Cyclodextrin | Known to improve solubility and stability |
| | | of many drugs and widely used in drug |
| | | delivery systems |

Among the above stabilizer candidates, excluding BHT, which is not suitable for sensitive skin, stability tests were conducted on the remaining three candidates. First, to test stability when mounted on a microneedle manufactured by a droplet extension (DEN) method using hyaluronic acid (HA), HA was used as a solidifying agent, and retinol raw material A, a stabilizer candidate material (tocopherol, sodium ascorbate, or cyclodextrin), and 100 mM of NaOH were added and mixed therein. In a total of three compositions, that is, compositions 1 to 3, the remaining ingredients (hyaluronic acid (HA), retinol raw material A, and NaOH) and composition ratios thereof are equal to each other except for the type of stabilizer candidate. Content % of HA was adjusted to a level for manufacturing a microneedle type using the DEN method, and the stabilizer was adjusted within a range of content % of a general excipient. After a weight of the mixture sample prepared with the above composition is adjusted to a constant level, the mixture sample was applied to DEN processes, which had already been commercialized at the time of application of the present disclosure and was disclosed in detail in the above-mentioned patents of the present applicant, and was solidified by applying an appropriate blowing condition.

The solidified microneedle simulation sample was put and sealed in an aluminum pouch and exposed to acceleration conditions (40±2° C., relative humidity 75±5%) for 4 weeks to perform an accelerated test. Retinol stability was evaluated at first, second, and fourth weeks. In the case of cyclodextrin-containing samples that had excellent stability up to a fourth week, retinol stability was evaluated by performing an accelerated test until an eighth week. Retinol stability evaluation was performed in the same manner as in Example 1.

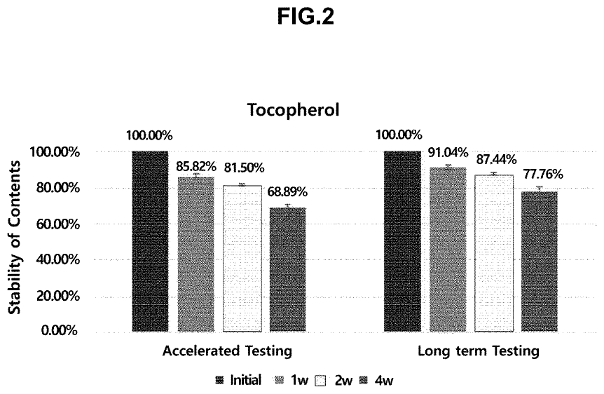

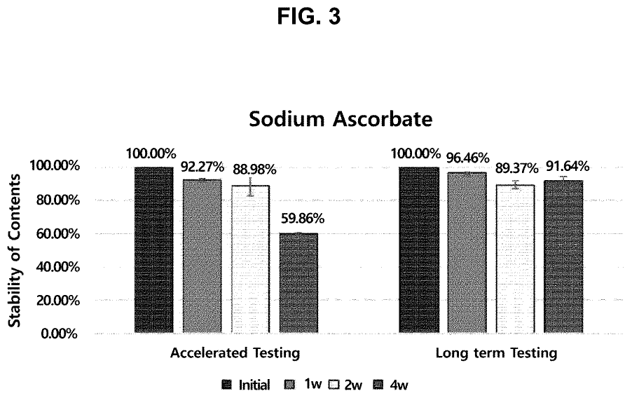

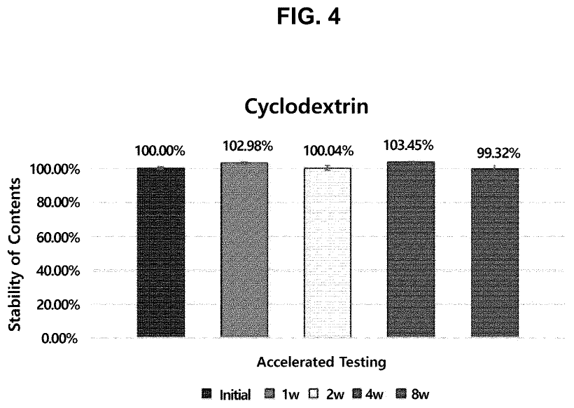

As a result, in the case of a microneedle formulation using hyaluronic acid (HA) as a solidifying agent, a stability effect of retinol was seen to be relatively high in the order of cyclodextrin >sodium ascorbate >tocopherol (refer to FIGS. 2 to 4).

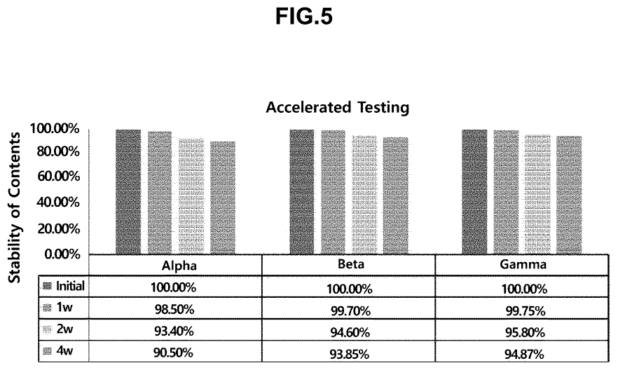

FIG. 5 shows results of an experiment evaluating the stability of the content of cyclodextrin included in a microneedle. As a result, excellent retinol stabilization was seen for all alpha, beta, and gamma cyclodextrins.

A stabilizing effect of cyclodextrin in the microneedle formulation as described above is determined to be achieved because an effect of the stabilizer varies depending on the formulation containing retinol.

The achievement of the present disclosure is to effectively preserve retinal, which easily loses the inherent skin improvement efficacy thereof due to formation of free radicals from oxidation or transformation from a trans form to a cis form, especially, a retinol component when included in a biodegradable microneedle. Even before the present disclosure, there were many attempts to stabilize a retinol component, but an effective method of stabilizing retinol in a biodegradable microneedle formulation as in the present disclosure is not identified from any attempt. An environment of retinol in a microneedle formulation is clearly differentiated from that in a water-in-oil (w/o), oil-in-water (o/w), or oil-in-water (o/w/o) type emulsion (the hyaluronic acid (HA) microneedle used in the present embodiment is a water-based formulation, and refer to the document [Katsunori Yoshida, et al., 1999, Stability of vitamin A in oil-in-water-in-oil-type multiple emulsions, Journal of the American Oil Chemists' Society, volume 76, pages 1-6]), and thus retinol stabilization methods in other environments may not be applied to biodegradable microneedle formulations.

In summary, when retinol was loaded in a hyaluronic acid (HA) microneedle, cyclodextrin was seen to achieve the best retinol stabilization, and furthermore, stability tests using cyclodextrin were performed not only at week 4 under accelerated conditions, but also at week 8 under accelerated conditions, and showed an amazing figure of 99.32% compared to the initial content.

1. A composition for a microneedle preparation, comprising:
retinol or a derivative thereof, cyclodextrin, and a biodegradable solidified material.

2. The composition for a microneedle preparation of claim 1, wherein the biodegradable solidified material is hydrophilic.

3. The composition for a microneedle preparation of claim 2, wherein the biodegradable solidified material is hyaluronic acid (HA) or pharmaceutically acceptable salt thereof.

4. A dissolvable microneedle manufactured using the composition of any one of claims 1 to 3.

5. The dissolvable microneedle of claim 4, wherein the microneedle is manufactured using a droplet extension (DEN) method.

6. The dissolvable microneedle of claim 5, wherein the microneedle is used to reduce wrinkles or increase skin elasticity.

7. A patch including the microneedle of claim 4.
